(19) United States
(12) Patent Application Publication
(10) Pub. No.: US 2019/0325053 A1
(43) Pub. Date: Oct. 24, 2019

(54) MEDICAL DATA CONVERSION DEVICE AND METHOD

(71) Applicant: Rifartek Technologies Co., Ltd., Hsinchu County (TW)

(72) Inventor: Tsung-Yi Tsai, Hsinchu County (TW)

(21) Appl. No.: 15/955,697

(22) Filed: Apr. 18, 2018

(51) Int. Cl. G06F 17/30; G16H 40/60; G16H 10/65; G16H 30/20

(52) U.S. Cl. CPC G06F 17/30569; G16H 30/20; G16H 10/65; G16H 40/60

(57) ABSTRACT

The medical data conversion device, after being connected to a medical instrument, detects an instrument data format used by the medical instrument, retrieves a format record corresponding to the instrument data format from a format database, extracting data from the medical instrument according to the format record, obtains user identity by an identification module, produces an intermediate data record integrating data from the format detection module, data extraction module, and the identification module by a conversion module. As such, data of inconsistent formats from various medical instruments may be integrated with user identity data for further processing, storage, and transmission.

### BACKGROUND OF THE INVENTION

### (a) Technical Field of the Invention

The present invention is generally related to medical data collection, and more particular to a conversion device capable of collecting data from various medical instruments and converting them for processing by a medical information system.

### (b) Description of the Prior Art

Conventionally, for a patient to go through a series of tests in the hospital, the paramedics conducting these tests have to check the patient's personal data, confirm the tests to perform, and record the test results, usually, on paper forms. There is a lot of unnecessary waiting time for the patients, affecting their levels of satisfaction. Human errors also seem inevitable, reducing the accuracy of these tests.

Furthermore, the patient has to hold on to the paper forms from the tests. Usually these paper forms do not have the patient's identification and, if they somehow lose the paper forms, the tests have to be repeated or the test results have to be manually copied from the instruments, causing significant inconvenience.

Therefore the digitization of medical data has become a main issue for medical facilities. Currently, the two most common systems in medical facilities are Healthcare/Hospital Information System (HIS) and Digital Imaging and Communications in Medicine (DICOM). The two systems however do not have a common and automatic interface with various medical instruments and a large amount of manual effort is still required to record and transmit medical data.

### SUMMARY OF THE INVENTION

A major objective of the present invention is to produce an intermediate data record of a standard format from data of inconsistent formats out of various medical instruments through format detection and conversion. Then, after integrated with user identity data, the intermediate data record may be further processed, stored, and transmitted. Human effort may be greatly reduced in the process.

To achieve the objective, the medical data conversion device is for electrically connecting a medical instrument, and includes a format database storing a number of format records; a format detection module inside the medical data conversion device and electrically connected to the format database, where the format detection module detects an instrument data format of an instrument data record output by the medical instrument and retrieves a corresponding format record from the format database; a data extraction module inside the medical data conversion device and electrically connected to the format detection module, where the data extraction module extracts data from the instrument data record according to the instrument data format specified in the format record; at least an identification module integrated with the medical data conversion device; and a conversion module producing an intermediate data record by integrating data from the format detection module, the data extraction module, and the identification module. When the medical data conversion device is applied, user personal information is collected via the identification module. Then, after various tests are conducted by different medical instruments, the formats of their data may be identified by the format detection module and the format database. Then data may be gathered by the data extraction module according to the relevant format records from the format database. An intermediate data record is produced by integrating user data and test result by the conversion module. In this process, due to data digitization and the present invention's self-adaptive algorithm in data integration, human error is greatly reduced and staff performance is enhanced. Subsequently, the convenience in reviewing or utilizing the medical data is significantly improved by converting the intermediate data record to required formats of HIS and other medical instruments by output and propagation modules.

Therefore, through the present invention, the existing medical information system's problems in involving significant human effort and high risk of human error are resolved.

The foregoing objectives and summary provide only a brief introduction to the present invention. To fully appreciate these and other objects of the present invention as well as the invention itself, all of which will become apparent to those skilled in the art, the following detailed description of the invention and the claims should be read in conjunction with the accompanying drawings. Throughout the specification and drawings identical reference numerals refer to identical or similar parts.

Many other advantages and features of the present invention will become manifest to those versed in the art upon making reference to the detailed description and the accompanying sheets of drawings in which a preferred structural embodiment incorporating the principles of the present invention is shown by way of illustrative example.

### BRIEF DESCRIPTION OF THE DRAWINGS

FIG. 1 is a perspective diagram showing a medical data conversion device according to an embodiment of the present invention.

FIG. 2 is a functional block diagram showing the medical data conversion device of FIG. 1.

FIG. 3 is a flow diagram showing a medical data conversion method according to an embodiment of the present invention.

FIG. 4 is a schematic diagram showing an application scenario of the medical data conversion device of FIG. 1.

FIG. 5 is a schematic diagram showing another application scenario of the medical data conversion device of FIG. 1.

FIG. 6 is a schematic diagram showing medical data being automatically propagated to another system.

FIG. 7 is a schematic diagram showing medical data being output to a printer.

FIG. 8 is a functional block diagram showing a medical data conversion device according to another embodiment of the present invention.

### DETAILED DESCRIPTION OF THE PREFERRED EMBODIMENTS

The following descriptions are exemplary embodiments only, and are not intended to limit the scope, applicability or configuration of the invention in any way. Rather, the following description provides a convenient illustration for implementing exemplary embodiments of the invention. Various changes to the described embodiments may be made in the function and arrangement of the elements described without departing from the scope of the invention as set forth in the appended claims.

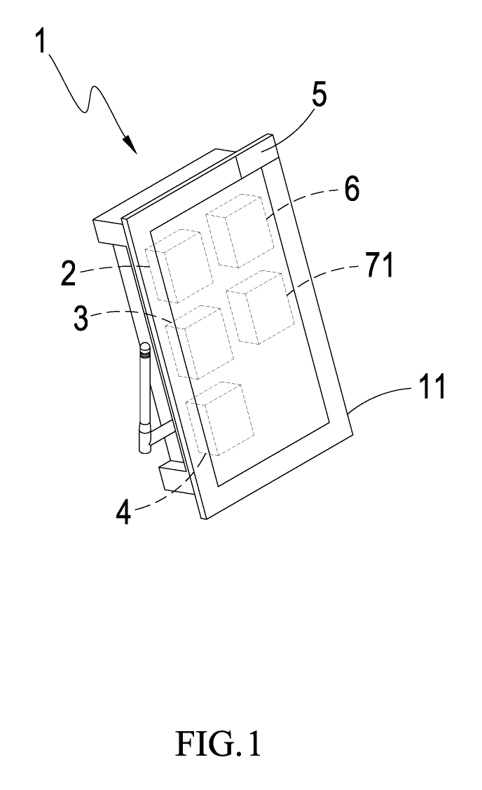

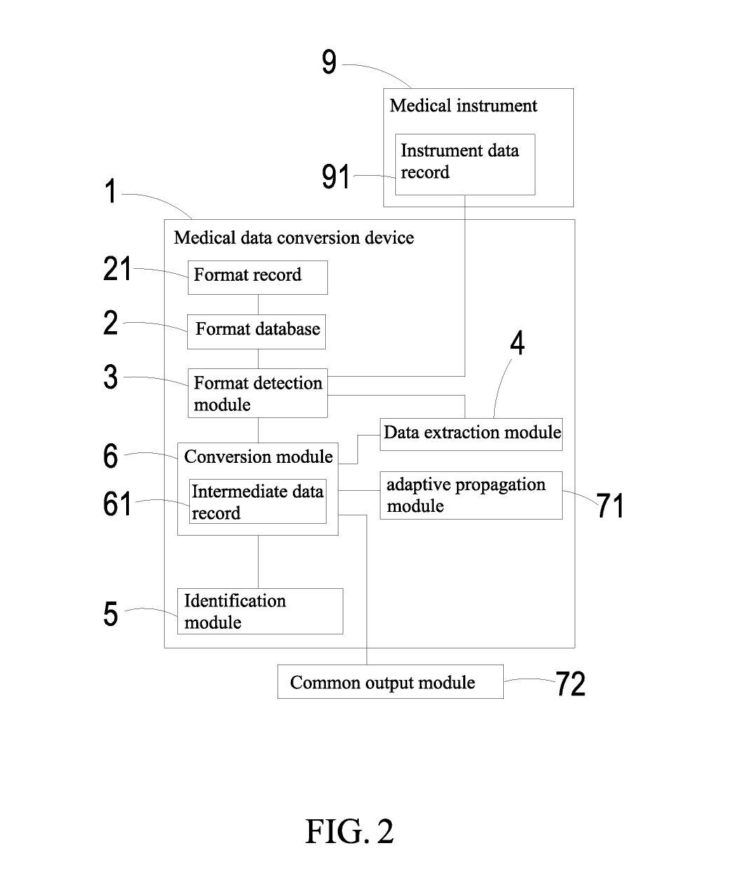

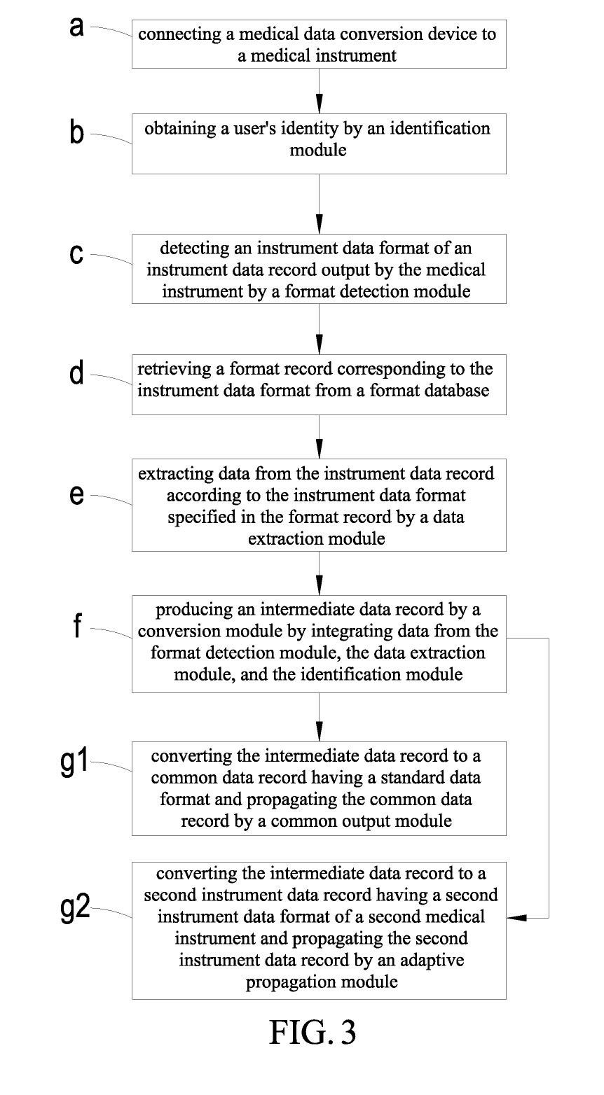

As shown in FIGS. 1 to 3, a medical data conversion device 1 according to an embodiment of the present invention is for electrically connecting a medical instrument 9 and is housed in a casing 11. The medical data conversion device 1 includes the following components.

A format database 2 is inside the medical data conversion device 1 stores a number of format records 21, each about how data are encapsulated in a particular data format.

A format detection module 3 is inside the medical data conversion device 1 and electrically connected to the format database 2. The format detection module 3 detects an instrument data format of an instrument data record 91 output by the medical instrument 9 and retrieves a corresponding format record 21 from the format database 2.

A data extraction module 4 is inside the medical data conversion device 1 and electrically connected to the format detection module 3. The data extraction module 4 extracts data from the instrument data record 91 according to the instrument data format specified in the format record 21.

At least an identification module 5 is integrated with the casing 11. The identification module 5 may be a card reader, a barcode reader, or a Radio Frequency Identification (RFID) reader.

A conversion module 6 integrates data from the format detection module 3, the data extraction module 4, and the identification module 5, and produces an intermediate data record 61.

An adaptive propagation module 71 is electrically connected to the conversion module 6. The adaptive propagation module 71 converts the intermediate data record 61 into a second instrument data record 91 of a second instrument data format used by a second medical instrument 9 and propagates the second instrument data record 91 to the second medical instrument 9.

A common output module 72 is electrically connected to the conversion module 6. The common output module 72 converts the intermediate data record 61 into a common data record of a standard data format such as one conforming to the Digital Imaging and Communications in Medicine (DICOM) protocol and outputs the common data record.

In an alternative embodiment, the medical data conversion device 1 may further include a touch screen for presenting test result from the medical instrument 9 or entering test result.

A medical data conversion method according to an embodiment of the present invention includes the following steps.

In step (a), a conversion device is connected to a medical instrument.

In step (b), a user's identity is obtained by an identification module.

In step (c), an instrument data format used by the medical instrument is detected by a format detection module.

In step (d), a format record is retrieved from a format database corresponding to the instrument data format.

In step (e), data is extracted from the medical instrument according to the data format specified in the format record by a data extraction module.

In step (f), an intermediate data record is produced by a conversion module by integrating data from the format detection module, the data extraction module, and the identification module.

In step (g1), the intermediate data record is converted to a common data record having a standard data format and the common data record is output by a common output module.

In step (g2), the intermediate data record is converted to a second instrument data record having a second instrument data format of a second medical instrument and the second instrument data record is propagated to the second medical instrument by an adaptive propagation module.

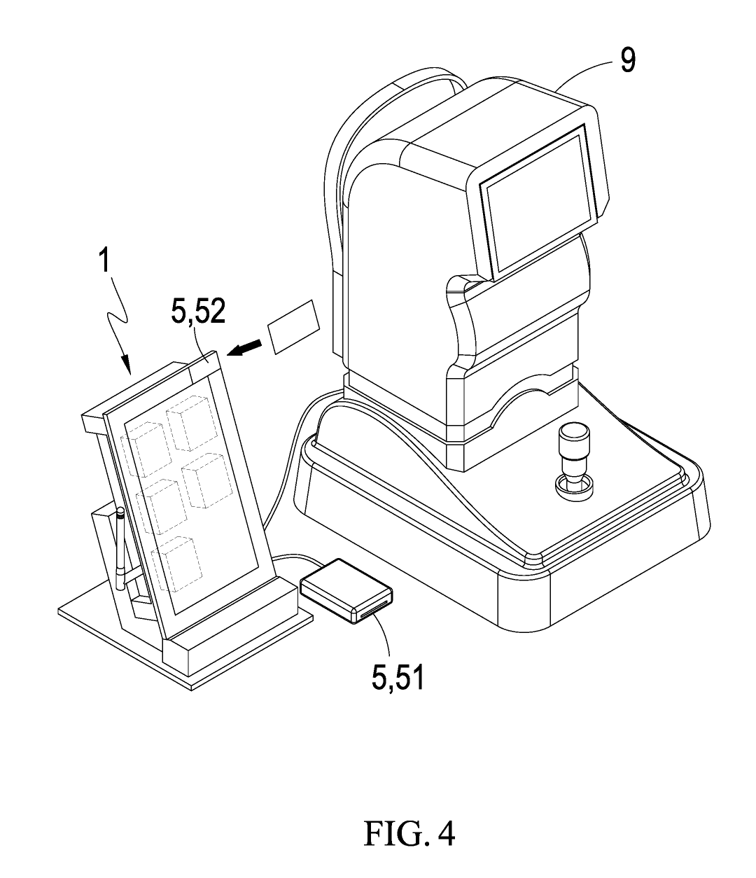

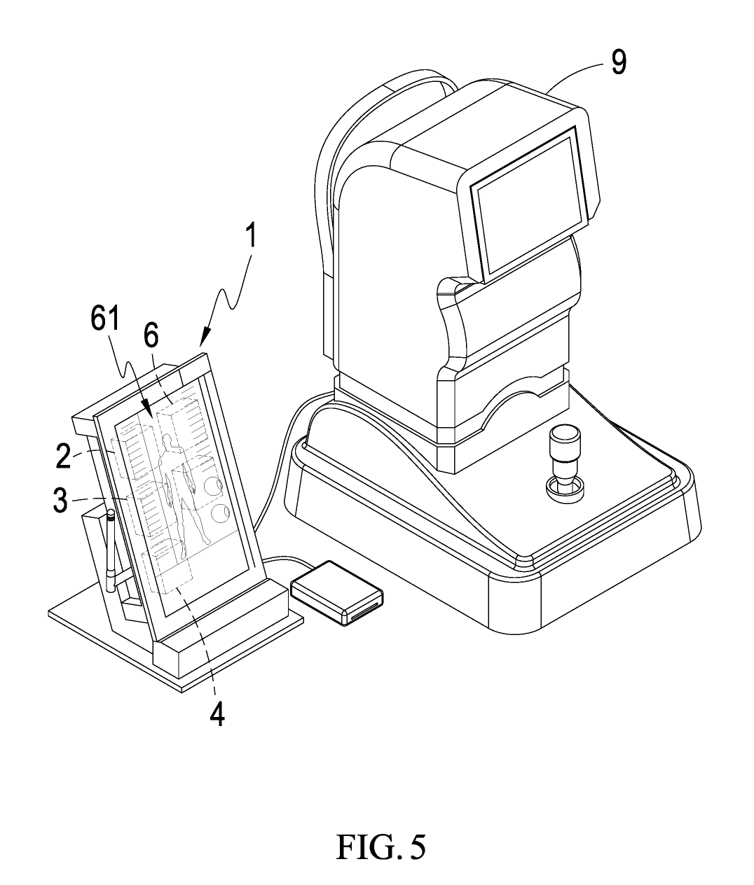

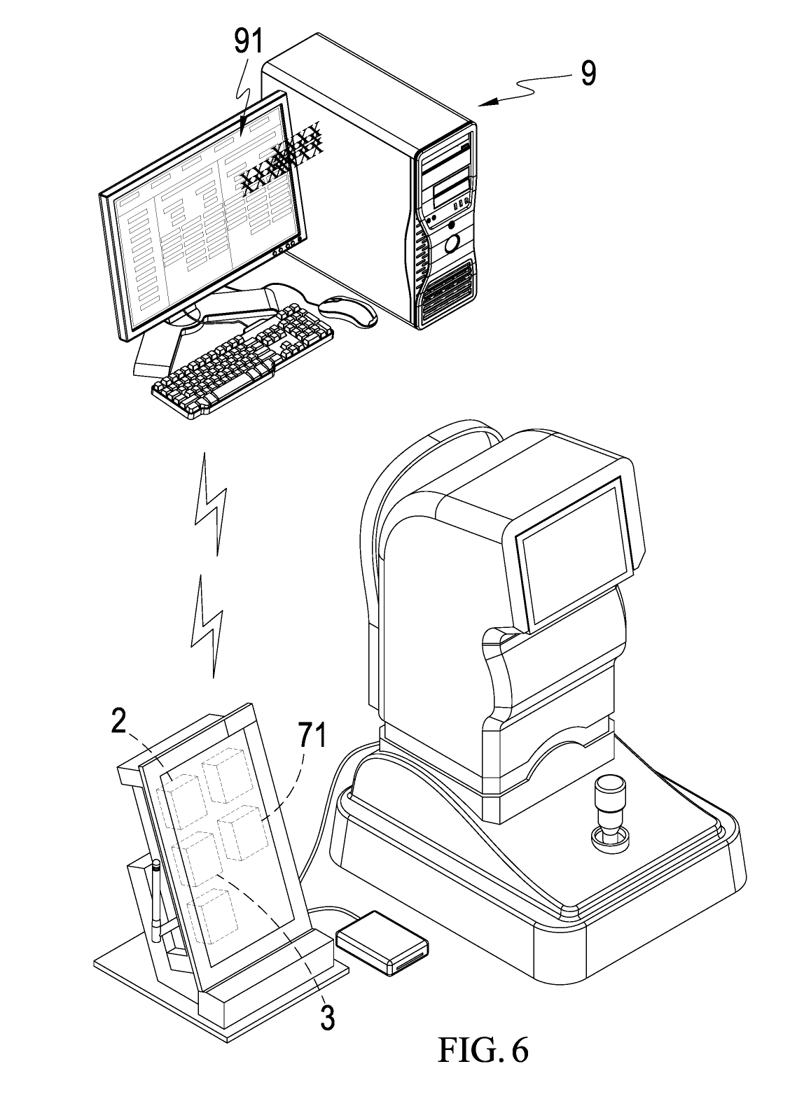

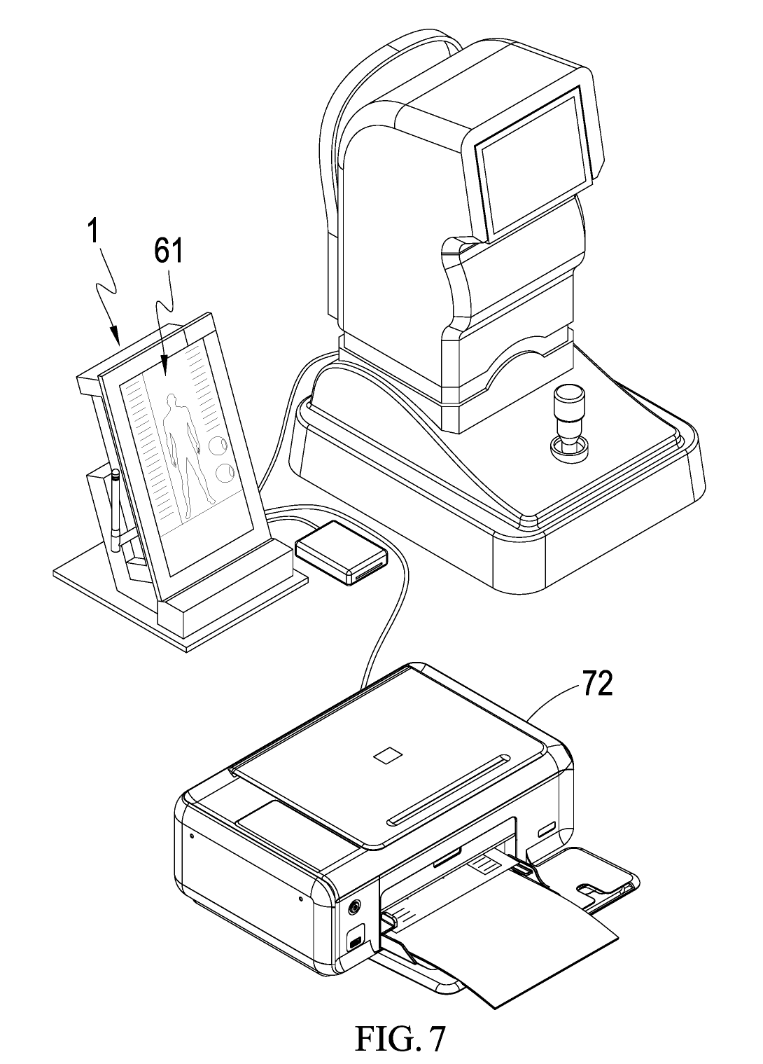

As shown in FIGS. 1 to 7, the medical data conversion device 1 of the present embodiment and the way it is hooked to the medical instrument is similar to a TV set top box. The major components described above are integrated with the casing 11 and has a form factor much smaller than most of the medical instruments 9. Once a medical instrument 9 having a proprietary data format is connected to the medical data conversion device 9, the medical instrument 9 is able to provide data in a standard format. To operate the medical data conversion device 1, user related data (such as personal information) is first obtained by the identification module 5 through a card reader 51 to access the chip on the health insurance ID card, a barcode reader to scan the barcode on the check-up form, or through RFID reader 52 to read an RFID tag. The identification module 5 may be inside the casing 11 or exposed on the casing 11. The present embodiment has both a card reader 51 and a RFID reader 52 as example. The step (b) of getting user identity can be conducted any time before the intermediate data record 61 is produced by the step (f). In other word, for example, an alternative embodiment of the medical data conversion method may involve the sequence of steps (a), (c), (d), (e), (b), W.

After test is conducted on the medical instrument 9, the result is stored in the medical instrument 9 and output in an instrument data record 91. The format detection module 3 compares the data format used by the instrument data record 91 against the format records 21 stored in the format database 2, and determines a format record 21 to be used in deciphering the instrument data record 91. The data extraction module 4, based on the format record 21, extracts any required data from the instrument data record 91. Then, the conversion module 6 integrates data from the format detection module 3, the data extraction module 4, and the identification module 5, and produces an intermediate data record 61. The intermediate data record 61 itself may be in a standard format such as the Digital Imaging Communications in Medicine (DICOM). This common communication protocol encapsulates user information, test related information in text and in image formats so that various medical instruments, servers, workstations, printers, and network equipment from different vendors may be integrated for medical data processing, storage, printing, transmission, such as in a Picture Archiving and Communication System (PACS).

As such, 80% of manual entry is avoided by the present embodiment. Even though a medical instrument 9 cannot accept data in DICOM format, the format detection module 3 and the format database 2 may detects the format used by the medical instrument 9, and the adaptive propagation module 71 collects the required data from the intermediate data record 61, prepares and propagates an instrument data record 91 in a format acceptable to the medical instrument 9. Especially when data is to be entered into Healthcare/Hospital Information System (HIS), a great deal of manual effort could be saved, staff's performance may be enhanced, and there is no need to modify Management Information System (MIS). If there is a need, the common output module 72 can also directly output the intermediate data 61 to another MIS system in formats such as JPG CSV. The operations of the common output module 72 (step g1) and the adaptive propagation module 71 (step g2) are optional, and are not required to be conducted in sequence.

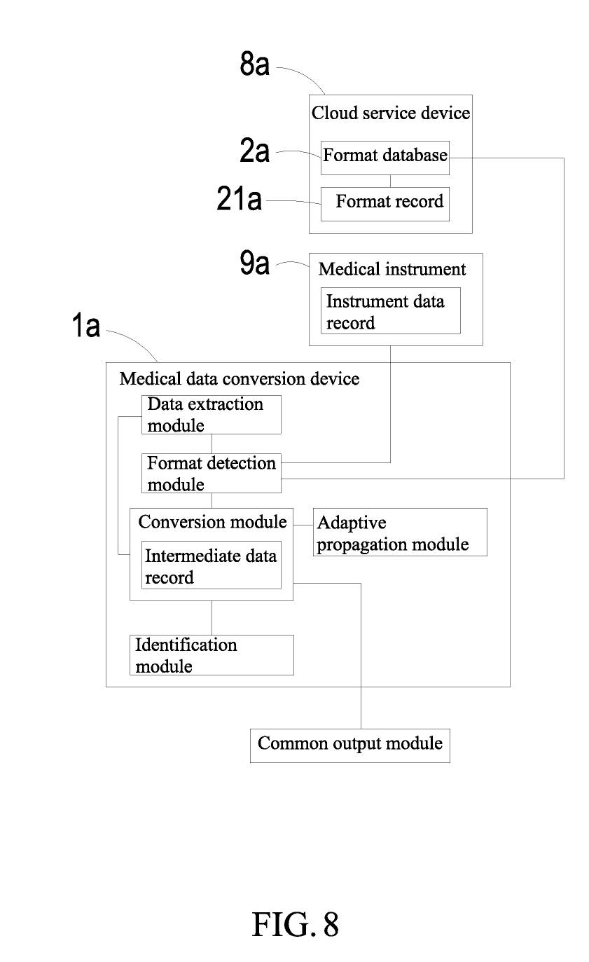

As shown in FIG. 8, a second embodiment of the present invention has the format database 2a not built in the medical data conversion device 1a, but stored in an external device or in a cloud service device 8a. In this way, format records 21a may be dynamically updated so as to support additional medical instruments 9a. In an alternative embodiment, there is a format database 2a inside the medical data conversion device 1a, and there is also a format database 2a in the cloud service device 8a. In this configuration, only the cloud format database 2a has to be maintained. Then, the medical data conversion device 1a may be linked to the cloud service device 8a and has its built-in format database 2a updated by the latest cloud format database 2a. As such, the convenience of maintaining the format database 2a and the performance of the medical data conversion device 1a are both enhanced.

The advantages of the present invention are as follows.

First, the medical data conversion device 1 may be adapted to all medical instruments 9.

Second, through the data extraction module 4 and the identification module 5, personal information and medical test results are all digitized and fast data delivery may be achieved.

Third, using a standard format such DICOM for the intermediate data record 61 conforms to the trend of medical information systems.

Fourth, using the adaptive propagation module 71 can quickly and automatically propagate intermediate data 61 into medical information system such as HIS.

While certain novel features of this invention have been shown and described and are pointed out in the annexed claim, it is not intended to be limited to the details above, since it will be understood that various omissions, modifications, substitutions and changes in the forms and details of the device illustrated and in its operation can be made by those skilled in the art without departing in any way from the claims of the present invention.

I claim:

1. A medical data conversion device for electrically connecting a medical instrument, comprising:
a format database storing a plurality of format records;
a format detection module inside the medical data conversion device and electrically connected to the format database, where the format detection module detects an instrument data format of an instrument data record output by the medical instrument and retrieves a corresponding format record from the format database;
a data extraction module inside the medical data conversion device and electrically connected to the format detection module, where the data extraction module extracts data from the instrument data record according to the instrument data format specified in the format record;
at least an identification module integrated with the medical data conversion device; and
a conversion module producing an intermediate data record by integrating data from the format detection module, the data extraction module, and the identification module.

2. The medical data conversion device according to claim 1, wherein the identification module is one of a card reader, a barcode reader, and a Radio Frequency Identification (RFID) reader.

3. The medical data conversion device according to claim 1, further comprising a common output module electrically connected to the conversion module, wherein the common output module converts the intermediate data record into a common data record of a standard data format and outputs the common data record.

4. The medical data conversion device according to claim 3, wherein the standard data format conforms to Digital Imaging and Communications in Medicine (DICOM) protocol.

5. The medical data conversion device according to claim 1, further comprising an adaptive propagation module electrically connected to the conversion module, wherein the adaptive propagation module converts the intermediate data record into a second instrument data record of a second instrument data format used by a second medical instrument, and propagates the second instrument data record to the second medical instrument.

6. The medical data conversion device according to claim 1, further comprising a touch screen for presenting test result from the medical instrument or entering test result.

7. The medical data conversion device according to claim 1, further comprising a casing, wherein the format database is inside the casing.

8. The medical data conversion device according to claim 1, wherein the format database is located in a cloud service device.

9. A medical data conversion method, comprising:
(a) connecting a medical data conversion device to a medical instrument;
(b) obtaining a user's identity by an identification module of the medical data conversion device;
(c) detecting an instrument data format of an instrument data record output by the medical instrument by a format detection module of the medical data conversion device;
(d) retrieving a format record corresponding to the instrument data format from a format database;
(e) extracting data from the instrument data record according to the instrument data format specified in the format record by a data extraction module of the medical data conversion device; and
(f) producing an intermediate data record by a conversion module of the medical data conversion device by integrating data from the format detection module, the data extraction module, and the identification module.

10. The medical data conversion method according to claim 9, wherein the identification module is one of a card reader, a barcode reader, and a Radio Frequency Identification (RFID) reader.

11. The medical data conversion device according to claim 9, further comprising:
(g1) converting the intermediate data record to a common data record having a standard data format and outputting the common data record by a common output module of the medical data conversion device.

12. The medical data conversion device according to claim 11, wherein the standard data format conforms to Digital Imaging and Communications in Medicine (DICOM) protocol.

13. The medical data conversion device according to claim 9, further comprising:
(g2) converting the intermediate data record to a second instrument data record having a second instrument data format of a second medical instrument and propagating the second instrument data record by an adaptive propagation module of the medical data conversion device to the second medical instrument.
